(19) United States
(12) Patent Application Publication
(10) Pub. No.: US 2002/0173475 A1
(43) Pub. Date: Nov. 21, 2002

(54) Methods to inhibit viral replication

(76) Inventors: Asim Dasgupta, Los Angeles, CA (US); Saumitra Das, Bangalore (IN); Narayan Baidya, San Jose, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 09/836,073

(22) Filed: Apr. 16, 2001

(51) Int. Cl. A61K 31/70; A01N 43/04; A61K 39/12; C07K 2/00; C07K 4/00; C07K 5/00; C07K 7/00; C07K 14/00; C07K 16/00; C07K 17/00; A61K 38/00; C07K 1/00

(52) U.S. Cl. 514/44; 530/300; 530/350; 424/186.1; 424/204.1

(57) ABSTRACT

Peptides having specific patterns of acidic and aromatic amino acids which inhibit viral infections are described. The peptides contain 15-18 amino acids and may be administered as such, in combination with additional antiviral agents and/or in the form of nucleotide sequences encoding them.

### TECHNICAL FIELD

The invention is directed to peptides that are effective to inhibit the replication of infective virus which utilize internal ribosome entry site (IRES) initiated translation, and/or to inhibit viruses that utilize the La protein in any phase of their life cycle. The peptides compete with the La protein and inhibit the utilization of various biochemical and physiological functions of La protein required for a productive virus life cycle. More specifically, the invention includes non-naturally occurring peptides which have advantageous properties in such inhibition.

### BACKGROUND ART

PCT publication WO96/11211, the contents of which are incorporated herein by reference describes methods to inhibit translation of messenger RNA at an internal ribosome entry site (IRES) based on an oligonucleotide which binds to a protein required for such translation. As described in the PCT publication, the oligonucleotide, designated “I-RNA” is based on the identification of an RNA of 60 nucleotides from yeast that exhibits the desired inhibitory properties. However, as further therein described, the I-RNA can also take the form of various mimics of the relevant portions of this yeast RNA such as the sequence of nucleotides 186-220 of polio virus or the sequence of nucleotides 578-618 of polio virus, and various other sequences. The significant aspect of this inhibitory oligonucleotide (I-RNA) is the nucleotide sequence itself, and its ability to bind. The endogenous protein to which I-RNA binds, designated human La autoantigen, is bound to the inhibitory oligonucleotide thus preventing the antigen's required binding to the mRNA and inhibiting the IRES from interacting with the ribosome. An 18-amino acid portion of this protein spanning positions 11-28 of the wildtype La autoantigen is shown to inhibit viral replication as described in PCT publication WO99/61613, also incorporated herein by reference. The amino acid sequence of the entire La autoantigen had been previously described by Chambers, J. C., et al., J. Biol. Chem. (1988) 253:18043-18061, also incorporated herein by reference. The mechanism whereby the 18-amino acid binding region of the La antigen (LAP) and the oligonucleotide described earlier inhibit translation of viral RNA is described in the above cited WO99/61613.

As previously described, a number of viruses, including picornavirus, hepatitis C virus, and others utilize the IRES mechanism to initiate translation. Since the above mentioned oligonucleotide and LAP inhibit this translation, they also inhibit viral replication and other essential viral functions. As demonstrated in these publications, the LAP is able readily to enter cells, and thus is in a position to effect this inhibition.

It has now been found that a family of peptides related to, but excluding LAP, have utility in inhibiting IRES initiated translation and thus inhibiting viral replication. These peptides are therefore useful in treating viral infections and in studies of the mechanism of viral infection.

### DISCLOSURE OF THE INVENTION

The invention is directed to a family of peptides that are useful as antiviral agents and as research tools for elucidating the mechanism of viral infection. These peptides inhibit IRES-initiated RNA translation.

Thus, in one aspect, the invention is directed to peptides of the formula

A¹ₙA²ₙA³ₙA⁴A⁵A⁶A⁷A⁸A⁹A¹⁰A¹¹A¹²A¹³A¹⁴A¹⁵A¹⁶A¹⁷A¹⁸ (1)

and its acylated and/or amidated forms thereof,

wherein each n is independently 0 or 1;

A¹, A², and A³ are each independently any amino acid;

A⁴, A¹², and A¹⁷ are independently acidic amino acids;

A¹³, A¹⁴, A¹⁵, and A¹⁸ are independently aromatic amino acids;

A⁵, A⁷, A⁸, A¹¹, and A¹⁶ represent any amino acid;

A⁶, A⁹, and A¹⁰ represent independently a basic amino acid or a polar neutral amino acid;

wherein each of said amino acids may be in the L form, racemic form, or D form.

Preferably, amino acids A⁵, A⁷, A⁸, A¹¹, and A¹⁶ are independently neutral, non-aromatic amino acids and are more preferably hydrophobic neutral, non-aromatic amino acids.

Preferably, A¹, A² and A³ are neutral, preferably non-aromatic amino acids and are preferably hydrophobic.

In additional aspects, the invention is directed to a method to inhibit IRES initiated translation and/or other viral functions by supplying the invention peptides to a cell-free system or to living cells infected with a virus which employs IRES initiated translation. The invention is also directed to methods to inhibit viral replication and to treat viral infection using the peptides of the invention or pharmaceutical or veterinary compositions thereof and is also directed to these pharmaceutical or veterinary compositions. Viral infections of plants can also be inhibited by the peptide of the invention and the peptides and nucleotide sequences encoding them are thus useful in agricultural and horticultural contexts as well. The peptides may be supplied as such, or generated from nucleotide sequences in situ.

The peptides and/or polynucleotides encoding them can be supplied in combination with other antiviral agents. In particular, combinations of the peptides of the invention or their encoding nucleotide sequences with the I-RNA described in WO96/11211 are particularly useful. However, other antiviral agents may be administered along with the peptide of the invention or the peptide of the invention in combination with I-RNA.

In another aspect, the invention is directed to antibodies which are specifically immunoreactive with the peptides of the invention. These antibodies, including immunoreactive fragments thereof are useful to purify the inhibitory protein of the invention and in assessing its level in various pharmaceutical, veterinary and agricultural compositions.

In addition, the peptides of the invention have the characteristic of preferentially associating with certain tissues, in particular, liver. Thus, the peptides can be employed as a specific delivery system for other compounds and compositions by fusing or otherwise associating the peptides of the invention with the material whose specific delivery is desired.

### MODES OF CARRYING OUT THE INVENTION

The peptides of the invention are of formula (1) set forth above wherein each amino acid is characterized as above defined. As noted, each of the three amino acid residues in positions 1, 2 and 3 may independently be present or absent. These amino acids are most preferably hydrophobic, but in general, any neutral amino acid could be substituted and, indeed, the amino acids in these positions are not important for activity and thus any amino acid may be employed. Preferably, A¹, A² and A³ are deleted from the peptide.

With one exception (A¹⁴ can also be neutral/polar in certain embodiments) the peptide is required to have acidic amino acids at A⁴, A¹² and A¹⁷ and aromatic amino acids at A¹³, A¹⁴, A¹⁵ and A¹⁸. The remaining positions are less critical.

It is conventional to group amino acids according to their general characteristics so that conservative substitutions can be made without altering the properties of the molecule. The simplest categories are acidic and basic amino acids. Acidic amino acids have a negative charge at physiological pH and are represented in the naturally-occurring amino acids by glutamic acid (glu or E) and aspartic acid (asp or D).

Similarly, basic amino acids have a positive charge at physiological pH and are represented in the naturally-occurring amino acids by lysine (lys or K), arginine (arg or R) and, to a lesser extent, histidine (his or H). The remaining amino acids are neutral, but these neutral amino acids can further be sub-classified. In particular, amino acids containing an aromatic system can be placed in the group of “aromatic” amino acids and are typified in the naturally occurring amino acids by tryptophan (trp or W), phenylalanine (phe or F) and tyrosine (tyr or Y).

Another subgroup of neutral amino acids are those which are polar, typified in the native amino acids by glutamine (gln or Q) and asparagine (asn or N). Sometimes serine (ser or S) and threonine (thr or T) are classified in this category as well due to the presence of OH.

Another subcategory of neutral amino acids are those which are clearly hydrophobic. Among naturally occurring amino acids, these include valine (val or V), leucine (leu or L), methionine (met or M) and isoleucine (ile or I). Sometimes included within this category are cysteine (cys or C) and alanine (ala or A) and even glycine (gly or G). Due to the small size of these amino acids, however, they are not as hydrophobic as isoleucine, valine and leucine.

The sole remaining naturally occurring amino acid, proline (pro or P) is difficult to classify, but it is clearly neutral.

As the peptides of the invention may be made synthetically as well as translated from a nucleotide sequence, there is no necessity to confine the amino acid residues to those which are encoded by the gene. Thus, the peptides may include such non-native amino acids as β-alanine (β-ala), 3-amino propionic acid, 2,3-diamino propionic acid (2,3-diap), 4-aminobutyric acid (4-aba), α-amino isobutyric acid (aib), sarcosine (sar), ornithine (orn), citrolline (cit), t-butyl alanine (t-bua), and others. These amino acids can be classified as acidic or basic according to their structures or may be neutral. The neutral forms may be classified as neutral/polar or neutral/hydrophobic or neutral/aromatic. An additional category is neutral/small which describes amino acids containing four carbons or less.

Again, because the peptides may be made synthetically, the native peptide linkage may be replaced with an isosteric linkage such as CH₂NH, CH₂S, CH₂CH₂, CH═CH (cis and trans), COCH₂, CH(OH)CH₂, and CH₂SO as representative examples. Means are well known in the art for synthesizing peptide-like molecules with these isosteric replacements.

Similarly, since synthetic methods are available for the synthesis of the compounds of the invention, the D forms of both the naturally occurring and non-naturally occurring amino acids can be employed as well as racemic mixtures thereof. A particularly preferred embodiment of the compound of formula (1) comprises amino acids in which all residues are in the D configuration. Such a compound is resistant to biodegradation and is thus particularly effective.

The genus described by the compounds of formula (1) specifically excludes the peptide represented by La antigen binding region (LAP) which is known in the art. This peptide has the formula labeled LAP in Table 1 below. Table 1 also sets forth specific muteins of the native LAP which are assigned numbers. It also shows the LAP amino acid sequences in homologs of the human LAP. Preferably these homologs are also excluded from the claimed genus, except when these peptides are provided in purified or isolated form or in the context of veterinary, pharmaceutical or agricultural/horticultural compositions.

As used herein, “isolated” refers to a state wherein the “isolated” material is removed from its natural surroundings. It may or may not be purified when it is isolated, but at least some of the components with which it is naturally associated have been removed or replaced.

TABLE 1

| Peptide | Sequence |
|---|---|
| LAP | AALEAKICHQIEYYFGDF |
| 702 | AALEAQICQQIEYYFGDF |
| 701 | AALQAKICHQIQYYFGQF |
| 761 | QQQEAKICHQIEYYFGDF |
| 762 | QQQEQKQCHQIEYYFGDF |
| 703 | AALEAKICHQIEQQQGDQ |
| 771 | AALEAKICHQIEYYQGDQ |
| 772 | AALEAKICHQIEQQFGDF |
| 631 | AALEAKICHQIEYYFGDQ |
| 632 | AALEAKICHQIEYYQGDF |
| 741 | AALEAKICHQIEQYFGDF |
| 633 | AALEAKICHQIEYQFGDF |
| MOUSE | ALEAKICHQIEYYFGDF |
| BOVINE | AALEAKICHQIEYYFGDF |
| XENOPUS | LDLDTKICEQIEYYFGDF |
| RAT | AALEAKICHQIEEYYFGDF |
| C. ELEGANS | DDADQRIIKQLEYYFGNI |
| MOSQUITO | VSKLEASTIRQEYYFGDA |
| DROSOPHILA | QERAIIRQVEYYFGDF |

In the compounds of the invention, described by formula (1), it is mandatory that A⁴, A¹² and A¹⁷ be acidic amino acids. Preferred embodiments of these amino acids are aspartic and glutamic; preferably A⁴ and A¹² are glutamic and A¹⁷ is aspartic. It is preferred that the optically pure form, either D or L is present in these positions.

It is also mandatory that A¹³, A¹⁴, A¹⁵ and A¹⁸ are aromatic amino acids, except A¹⁴ can be neutral/polar. Preferred aromatic amino acids are phenylalanine and tyrosine. Preferably A¹³ and A¹⁴ are tyrosine and A¹⁵ and A¹⁸ are phenylalanine. However, these residues can be rearranged such that, for example, A¹³ and/or A¹⁴ are phenylalanine and A¹⁵ and/or A¹⁸ are tyrosine, or all such residues may be tyrosine or all phenylalanine.

The remaining amino acids in compounds of formula (1) are less critical than those described above. In particular, however, it is preferred that the residues at A⁶ and/or A⁹ be basic amino acids or neutral polar amino acids. A neutral polar amino acid is preferred at A¹⁰, but a basic amino acid may also be substituted. Any of these positions may be replaced, though it is less preferred, with asparagine. Even less preferred, although permitted within the scope of the invention, is replacement of these positions by glycine, alanine, threonine or serine.

A⁷ and A³, when present, are preferably hydrophobic amino acids, most preferably leucine, isoleucine, valine or methionine, most preferably leucine, isoleucine or valine. Preferably A¹ and A², if present, and A⁵ are hydrophobic but small, and are, for example, alanine, cysteine, or glycine, preferably alanine. A¹⁶ is preferably glycine, but may also be a small amino acid such as alanine or serine or threonine. Preferably A⁸ is cysteine but a similar amino acid may also be substituted, such as serine, threonine, alanine or glycine.

Particularly preferred embodiments of the compounds of formula (1) are peptide 702, peptide 761, and peptide 633.

Peptide 701 is also preferred.

The peptides of the invention, as mentioned above, may be made using standard synthetic techniques either solution or solid phase based techniques as is well understood in the art, or, if composed of L-isomers of gene-encoded amino acids may be made recombinantly. Recombinant production of these peptides may employ synthetic polynucleotides which are readily synthesized using commercially available instrumentation. The coding region, optionally supplemented with a leader sequence to effect secretion can then be expressed in host cells either previously operably linked to control sequences to effect expression or using endogenous control sequences for this purpose. The peptide can be produced in a variety of cells, including prokaryotic and eukaryotic cells such as yeast and mammalian cells. The peptides could, if desired, be produced transgenically in animals or plants.

The peptides of the invention, however produced, may be supplied in forms wherein the N-terminus is acylated, for example, acetylated, and/or the C-terminus is amidated, e.g., as the carboxamide, or as the amide obtained by reaction of the carboxylic acid residue with an alkyl or dialkyl amine. Such alkyl or dialkyl amine preferably has six carbons or less.

In addition to derivatizing the N or C terminus, or other reactive groups on the site chains of the peptide, the amide linkages of the peptide itself may be substituted by isosteres such as CH₂NH or CH₂O, CH₂CO and the like. Methods for synthesizing such pseudo-peptides are also well understood in the art.

The peptides of the invention may also be supplied as their pharmaceutically acceptable salts, or may be lipidated or glycosylated.

The peptides of the invention, including the acylated and/or amidated forms thereof may be formulated into pharmaceutical or veterinary compositions for use in antiviral therapy. Such compositions appropriate for peptides may be found in Remington's Pharmaceutical Sciences, latest edition, Mack Publishing Co., Easton, Pa., incorporated herein by reference. The compounds may be formulated for oral, transdermal, transmucosal, or other routes of entry or may be injected. Standard compositions are understood in the art to include tablets, capsules, solutions, powders, syrups, lotions, and the like. The excipients contained in such formulations may include a variety of carriers including liposomes and surfactants. Appropriate formulations and routes for administration of peptides will be understood by the ordinary practitioner. One particularly favorable formulation includes coupling the peptide with a targeting agent or an agent, such as the tat protein which is able to facilitate entry of coupled proteins into cells. Dosage levels will vary with the condition of the subject, the severity of viral infection, and the judgment of the attending practitioner. The optimization of formulation and dosage is well within ordinary skill of a physician or veterinarian as the case may be.

Among the many pharmaceutical/veterinary compositions possible for the peptides of the invention are various slow-release systems based on polymers which may or may not degrade in order to release the active ingredient at an ordered rate or other matrices for providing a constant supply of the peptide.

For agricultural application, formulations suitable for providing the peptide to affected or potentially affected plants are employed. Such compositions may include soil treatment compositions, sprays, or more precise application to individual plants may be employed.

In addition to the use of the compounds of the invention to treat viral infection in animal subjects or plants, the peptides may be used to study the mechanism of viral infection and replication in a laboratory context. Thus, their effect on various modified forms of viral mRNA, for example, can be evaluated in a cell-free system or in cell culture. Means for using such peptides in these contexts are well known.

In all of the above contexts, formulations intended for treatment may also be administered using the encoding nucleic acids, either administered as naked DNA or included in expression systems. The construction of expression systems for a wide variety of host cells is well understood in the art; suitable promoters, enhancers, termination signals, and the like that perform significant functions in expression have been developed that are appropriate for various mammalian cells, avian cells, plant cells, yeast cells, insect cells, and the like. The promoters may be constituitive or inducible. Thus, the expression system is designed for the subject to be treated. For mammalian subjects, for example, suitable promoters would include the metalathionin promoter, various viral-derived promoters such as an SV40 promoter, and the like. The expression systems may include viral vectors which are especially convenient for administration to mammalian subjects. Particularly preferred embodiments include viral vectors, such as adenoviral vectors, or other viral vectors, such as retroviral vectors as is understood in the art. The vectors may be made to be conditionally replicating so that only in cells infected with virus do the vectors themselves replicate and produce protein.

Vectors may be designed for replication and expression in response to viral infection or in response to other external chemical or physical inducing signals. The vectors may also contain homologous sequences for integration of the appropriate expression system into the host cell's genome or may be otherwise designed to replicate and be passed to the daughter cells when the cells divide. A large number of suitable vectors is known in the art, including, for example, self-inactivating lentiviral vectors adapted for transduction of CD 34(+) cells, and mixtures of adenoviral vectors with wild type adenovirus.

As noted above, the peptide may be administered in combination with other antiviral agents, including I-RNA, which is described in PCT publication WO96/11211. By “I-RNA” is meant any nucleotide sequence which mimics the inhibitory effects of the endogenous yeast inhibitory RNA described therein. A number of nucleotide sequences comprising such mimics are described in the PCT publication. It should be emphasized that the inhibitory affect of the I-RNA is a function of the nucleotide sequence per se; therefore, the I-RNA can be administered as RNA, DNA, or a substitute nucleic acid backbone such as the peptide nucleic acids that are well known in the art. The “I-RNA” thus includes nucleotide sequences coupled along any supporting backbone. Indeed, the bases which form the nucleotide sequence need not be the naturally occurring A, T (U) G, C but may also include modified forms of these bases that retain the essential complementary features of the native nucleotides.

A number of combination compositions can be formulated which are useful in the methods of the invention to control viral infection. These include the combination of the invention peptide with I-RNA as defined above. The combination of an expression system for the invention peptide in combination with I-RNA, the peptide of the invention or an expression system therefor combined with a different antiviral agent such as acyclovir, an antisense antiviral sequence or an expression system therefor, a ribozyme which targets viral RNA or an expression system therefor, or any other antiviral agent or combination of antiviral agents. These antiviral agents may also be used in combination with the combination of I-RNA and the peptide of the invention.

In still another aspect, the invention is directed to antibodies that are specifically immunoreactive with the invention peptides. As noted above, these antibodies are useful in assessing the concentration of the peptide in formulations, as well as monitoring the levels of the peptides of the invention after administration. As defined herein, “antibodies” or “immunoglobulins” includes fragments such as Fab, Fab′, and the like that retain the immunospecificity of complete antibodies and also includes recombinant forms of the antibodies, including single chain forms such as Fᵥ forms. Methods to generate antibodies to the peptide of the invention are well-known in the art; immunospecificity can be assured by the results of binding assays with the protein of the invention as contrasted with the results of binding assays to similar but nevertheless different peptides. A binding differential of 10-fold, preferably 100-fold and more preferably 1,000-fold is indicative of such specificity.

The antibodies immunospecific for the invention may be polyclonal or monoclonal and recombinant or generated by immunization. They can be modified to be species-specific, using methods well-known in the art. Thus, if desired, for example, these antibodies could be humanized.

The immunoglobulin specific for the peptides of the invention as defined above can be coupled to solid supports and used to purify the peptides of the invention.

Although the invention peptides appear to home to certain tissues, in particular to liver, they may themselves be targeted to desired locations by coupling them to additional protein or other compositions which are specific to particular organs or tissues. Thus, the peptides may be coupled to ligands which bind desired target receptors or to compounds which are reactive, for example, with cell surface proteins or tumor associated antigens.

Nevertheless, as the invention peptides do home to liver tissue, they may themselves be used as delivery systems to transport other compounds to these locations. Thus, another aspect of the invention is the use of the invention peptides as delivery systems for additional compounds.

### EXAMPLES

The following examples are intended to illustrate but not to limit the invention.

### Preparation A

### Preparation of Test Peptides

Using Stratagene's QuikChange™ site-directed mutagenesis kit (#200518), amino acid changes were introduced into a wild-type cDNA clone of La (generously provided by Dr. Jack Keene, Duke University, North Carolina) (Chambers, J. C., et al., Proc. Natl. Acad. Sci. USA (1985) 82:2115-2119). The recombinant proteins were expressed in E. coli (BL21 (DE3) pLysS) by inducing for 4 hours with 0.4 mM IPTG. The cells were lysed by sonication and the lysate was centrifuged (12,000×g) for 30 minutes at 4° C. The supernatant was treated with streptomycin sulfate (3% final concentration) and centrifuged as above. The supernatant was dialyzed overnight at 4° C. against Buffer A (25 mM Tris pH 8.0, 100 mM NaCl) and then charged onto a DEAE Sephacel column equilibrated with Buffer A. The flow through was collected and separated using FPLC with a heparin agarose column and 0 to 1 M NaCl gradient. The fractions containing purified La or La mutants were pooled and dialyzed against BufferA.

For experiments testing the ability of the peptide to enter cells, peptides were FITC-labeled using Molecular Probe's FluoReporter™ FITC protein labeling kit (F-6434) according to manufacturer's instruction with a slight modification. All the peptides were synthesized and purified to >95% homogeneity by Biosynthesis (http://www.biosyn.com). The peptides were dissolved in 100 mM Tris-HCl, Ph 8.0 at 5 mg/ml and then diluted to 1 mg/ml using nuclease free water for subsequent use in translation assays. For FITC-labeling, the peptides were dissolved in PBS, pH 8.0.

### Example 1

### Effect of Various Peptides on In Vitro Translation

In these experiments, p2CAT (Coward, P., et al., J. Virol (1992) 66:286-285) plasmid DNA was linearized with BamHI and in vitro transcribed with SP6 RNA polymerase. The transcribed mRNA was purified using phenol:chloroform extraction and EtOH precipitation. PV 5′ UTR, a clone of the 5′ UTR of poliovirus (Das, S., et al., J. Virol. (1994) 68:7200-7211), was linearized with HindIII and transcribed with T7 RNA polymerase in the presence of α-³²P UTP (3000 Ci/mmole). The radiolabeled RNA was purified using Quick Spin RNA columns (Roche).

HeLa cells were grown in monolayers in minimum essential medium (GIBCO/BRL) supplemented with 10% fetal bovine serum. HeLa cell lysates were prepared as previously described (Coward, et al., supra; Rose, J. K., et al., Proc. Natl. Acad. Sci. USA (1978) 75:2732-2736). In vitro translation of p2CAT using HeLa cell extract was performed essentially as described elsewhere (Rose, et al., supra). One microgram of the in vitro transcribed p2CAT RNA was translated in 80 μg of HeLa cell extract in a 25 μl reaction mixture in the presence of 25 μCi of ³⁵S methionine and 40 U of RNAsin (Promega). Peptides were added at 20, 40 and 60 μM concentration per reaction. As a negative control, 3 μl of diluted (1:5) 100 mM Tris-HCl, pH 8.0 was also tested.

The results for the peptides shown in Table 1 are as follows: Peptide 702, wherein the lysine at A⁶ and histidine at A⁹ were replaced by the neutral polar amino acid glutamine, was effective at inhibiting translation of the CAT reporter protein at concentrations of 40 and 60 μM; however, peptide 701 wherein the acidic residues at positions A⁴, A¹² and A¹⁷ are all replaced by the neutral polar amino acid glutamine, lost the ability to inhibit translation at these concentrations.

Peptide 761, where A¹, A², and A³ were replaced by the polar neutral amino acid glutamine was inhibitory at 60 μM and slightly inhibitory at 40 μM. However, when, in addition to this replacement at A¹-A³, the alanine at A⁵ and the isoleucine at A⁷ were replaced by glutamine, the ability of the peptide to inhibit translation was lost.

In similar experiments, using peptides 703, 771 and 772, where one or more of the aromatic amino acids at position A¹³ and beyond were replaced by glutamine, inhibitory activity was also lost. In these embodiments, at least two of the aromatic residues in A¹³, A¹⁴, A¹⁵ and A¹⁸ were thus replaced.

If only one replacement was made, as in peptide 631, 632, 741 and 633 again the ability to inhibit translation was diminished. However, peptides 633 and 631 were still able to inhibit at higher levels of concentration; indeed peptide 631 inhibited at concentrations of 40 μM and 60 μM and 633 at 60 μM. In peptide 631, only A¹⁸ was replaced by glutamine; in peptide 633 only A¹⁴ was thus replaced.

Thus, although A¹³, A¹⁴, A¹⁵ and A¹⁸ are mandated generally as aromatic amino acid residues, included within the scope of the invention are embodiments where A¹⁴ or A¹⁸ is replaced by a neutral amino acid, preferably other than a small neutral amino acid.

### Example 2

### Ability to Bind mRNA per se

For this analysis, ³²P-labeled PV 5′ UTR RNA (1×10⁶ cpm) probes were incubated with recombinant wild-type La or its mutants for 10 minutes at 30° C. After binding, the reaction was completed as described in (Banerjee, R., et al.) “In-vitro Replication of RNA Viruses,” p. 158-164 in A. Cann (ed.), RNA Viruses: A Practical Approach (2000) Oxford University Press, New York.

The labeled PV 5′ UTR RNA was UV crosslinked to 500 ng, 1 μg or 1.5 μg of wild-type La antigen or two peptides 771 or 772. After RNase digestion, the protein nucleotidal complexes were analysis on SDS polyacrylamide. The results showed dramatically reduced binding for both peptides.

In a similar experiment, similar results were obtained for peptide 741 where A¹³ is converted from tyrosine to glutamine. Only slightly reduced binding was observed when A²⁵ was changed from phenyl alanine to glutamine and when the tyrosine at A²⁴ was changed to glutamine.

### Example 3

### Cell Entry and Retention

For these experiments, FITC-labeled peptides were used. Labeling of the peptide was described in Preparation A above. After labeling, the peptides were purified using Quick Spin RNA columns (Roche). HeLa or Huh-7 cells grown in slide chambers were incubated with 5 μM of each peptide overnight. (The hepatocellular carcinoma cells (Huh-7) were grown in RPMI medium (GIBCO/BRL) supplemented with 10% fetal bovin serum.) The cell membranes were subsequently stained for 20 minutes with a 1:200 dilution of DiIC₁₈ (Molecular Probes) at a working concentration of 1 mg/ml and then washed 3 times with PBS. The cells were layered with 25 μl of Gelvatol and covered with glass coverslips. The cells were analyzed in a Leitz confocal laser scanning microscope system using a 100× oil immersion lens.

In general, the results showed that the ability of the peptides to enter and be retained in the cells paralleled their activity to inhibit translation. A summary of the results for the various peptides tested in shown in Table 2.

TABLE 2

| Peptide | Mutation | Activity | Cell Entry |
|---|---|---|---|
| LAP | AALEAKICHQIEYYFGDF | + | +++ |
| 702 | AALEAQICQQIEYYFGDF | + | +++ |
| 701 | AALQAKICHQIQYYFGQF | − | + |
| 761 | QQQEAKICHQIEYYFGDF | + | + |
| 762 | QQQEQKQCHQIEYYFGDF | − | − |
| 703 | AALEAKICHQIEQQQGDQ | − | − |
| 771 | AALEAKICHQIEYYQGDQ | − | − |
| 772 | AALEAKICHQIEQQFGDF | − | − |
| 741 | AALEAKICHQIEQYFGDF | − | − |
| 633 | AALEAKICHQIEYQFGDF | + | +++ |
| 632 | AALEAKICHQIEYYQGDF | − | − |

1. A compound of the formula
A¹ₙA²ₙA³ₙA⁴A⁵A⁶A⁷A⁸A⁹A¹⁰A¹¹A¹²A¹³A¹⁴A¹⁵A¹⁶A¹⁷A¹⁸ (1)
and acylated and/or amidated forms thereof,
wherein each n is independently 0 or 1;
A¹, A², and A³ are each independently any amino acid;
A⁴, A¹², and A¹⁷ are independently acidic amino acids;
A¹³, A¹⁴, A¹⁵, and A¹⁸ are independently aromatic amino acids;
A⁵, A⁷, A⁸, A¹¹, and A¹⁶ represent any amino acid;
A⁶, A⁹, and A¹⁰ represent independently a basic amino acid or a polar neutral amino acid;
wherein each of said amino acids may be in the L form, racemic form, or D form:

2. The compound of claim 1 wherein all amino acids are gene encoded.

3. The compound of claim 1 wherein all linkages between Aⁱ subunits are amide linkages.

4. The compound of claim 1 where all of Aⁱ are in the D form.

5. The compound of claim 1 wherein all of Aⁱ are in the L form.

6. The compound of claim 1 wherein each of A⁴, A¹² and A¹⁷ is independently aspartic or glutamic.

7. The compound of claim 1 wherein each of A¹³, A¹⁴, A¹⁵ and A¹⁸ is independently phenylalanine or tyrosine.

8. The compound of claim 1 wherein A⁸ is cysteine.

9. The compound of claim 1 wherein each of A⁶, A⁹ and A¹⁰ is independently lysine, histidine, arginine, glutamine, or asparagine.

10. The compound of claim 1 which is selected from the group consisting of AALEAQICQQIEYYFGDF, AALQAKICHQIQYYFGQF, QQQEAKICHQIEYYFGDF and AALEAKICHQIEYQFGDF.

11. The compound of claim 1 which is in isolated or purified form and is selected from the group consisting of ALEAKICHQIEYYFGDF, AALEAKICHQIEYYFGDF, LDLDTKICEQIEYYFGDF, AALEAKICHQIEEYYFGDF, DDADQRIIKQLEYYFGNI, VSKLEASTIRQEYYFGDA and QERAIIRQVEYYFGDF.

12. A pharmaceutical, veterinary or agricultural/horticultural composition which comprises the compound of claim 1 along with a suitable excipient.

13. A nucleic acid molecule comprising a nucleotide sequence encoding the compound of claim 2.

14. A recombinant expression system comprising a nucleotide sequence encoding the compound of claim 2 operably linked to control sequences effective for its expression.

15. A recombinant host cell modified to contain the expression system of claim 14.

16. The recombinant host cell of claim 15 wherein said expression system is integrated into the genome of said host cell.

17. A method to produce the compound of claim 2, which method comprises effecting expression of said compound from the expression system of claim 14.

18. The expression system of claim 14 which is included in a viral vector.

19. The viral vector of claim 18 which is an adenoviral vector or a retroviral vector.

20. A method to treat viral infection in a plant or animal subject which method comprises administering to said subject an antivirally effective amount of the compound of claim 1.

21. The method of claim 20 wherein said method further comprises administering at least one additional antiviral agent.

22. The method of claim 21 wherein said administering of the compound and said at least one additional antiviral agent is substantially simultaneous.

23. The method of claim 21 wherein said administering of the compound of claim 1 and said at least one antiviral compound is sequential.

24. The method of claim 21 wherein said additional antiviral compound is I-RNA.

25. A method to treat viral infection in a plant or animal subject, which method comprises administering to said subject an antivirally effective amount of a nucleotide sequence encoding the compound of claim 2.

26. The method of claim 25 wherein said nucleotide sequence is comprises in an expression system compatible with the cells of said subject.

27. The method of claim 25 wherein said method further comprises administering at least one additional antiviral agent.

28. The method of claim 27 wherein said administering of the compound and said at least one additional antiviral agent is substantially simultaneous.

29. The method of claim 27 wherein said administering of the compound of claim 1 and said at least one antiviral compound is sequential.

30. The method of claim 27 wherein said additional antiviral compound is I-RNA.

31. A method to deliver a compound selectively to the liver, which method comprises administering to a subject containing a liver a desired compound coupled to the compound of claim 1.

32. Antibodies specifically immunoreactive with the compound of claim 1.

33. The antibodies of claim 32 which are immunospecific fragments.

34. The antibodies of claim 33 which are monoclonal antibodies.

35. A method to purify the compound of claim 1, which method comprises contacting a sample containing said compound with antibodies specifically immunoreactive therewith, said antibodies coupled to a solid support.
